(19) United States
(12) Patent Application Publication
(10) Pub. No.: US 2026/0240708 A1
(43) Pub. Date: Aug. 20, 2026

(54) A Method and Apparatus for Bone Conditioning

(71) Applicant: Nebotek Ltd, Nicosia (CY)

(72) Inventors: Panayiotis Philimis, Nicosia (CY); Nicos Raftis, Nicosia (CY); Neophytos Hadjichristou, Nicosia (CY); Marios Kokonas, Nicosia (CY); Antonis Hadjiantonis, Nicosia (CY)

(21) Appl. No.: 19/148,186

(22) PCT Filed: Jan. 16, 2024
(86) PCT No.: PCT/EP2024/050925

§ 371 Date: Jul. 15, 2025

(30) Foreign Application Priority Data
Jan. 16, 2023 (GB) 2300603.4

(51) Int. Cl. A61H 1/00; A61H 15/00

(52) U.S. Cl. CPC A61H 1/006; A61H 15/0078; A61H 2205/106

(57) ABSTRACT

The method for non-therapeutic conditioning of a user's bone is provided. The method comprises assessing the state of the bone and generating, based upon the state of the bone, a treatment plan for conditioning the bone by forming microcracks in the bone. The method further comprises including localized stresses on at least a part of the bone in accordance with the treatment plan. The localized stresses are induced by a conditioning device comprising a contact device and an actuator. A controller is configured to control the conditioning device in accordance with the treatment plant to mechanically induce the localized stresses on the bone.

### TECHNICAL FIELD

The present disclosure is directed towards a method and an apparatus for bone conditioning. More specifically, the present disclosure relates to a method and corresponding apparatus for conditioning of the shin of martial arts athletes.

### BACKGROUND

Shin conditioning is a process for improving the performance of an athlete in many martial arts. Traditionally shin conditioning comprises three steps: (a) impacting the shin to cause microcracks in the bone, (b) allowing healing to occur and (c) repeating steps 1 and 2 every 3-5 days for several months.

FIGS. 1a and 1b show views of the lower leg illustrating the tibia (shin) and fibula. When referring to shin conditioning, we refer to the process of strengthening the part 101 of the tibia bone 10 that would typically receive impacts during a fight. FIG. 1c illustrates a typical section of the lower leg showing tibia, fibula, muscles, and skin. The impact on the tibia bone 10 is shown with a dotted line 102.

Shin conditioning has several benefits including, strengthening of the bone, conditioning for greater pain tolerance and greater confidence during a fight.

This is an ancient practice that requires discipline and dedication. In traditional practice, when a martial arts fighter strikes a hard surface, whether that be a heavy bag or an opponent in the ring, their bones suffer microfractures. The body recognises these microfractures as weak points and then prioritises them for repair by creating calcium deposits on top of the damaged bone tissue. This is referred to as ‘ossification’. Commonly, martial arts athletes practicing shin conditioning using traditional methods by kicking a vertical round surface.

Bone is a complex material composite of collagen, and mineral, calcium phosphate. The relationship between the mineral and collagen phases affects mechanical properties of the bone. Mechanical properties are primarily strength, hardness, toughness, and elasticity. The mechanical properties are determined by a process of bio-mineralisation, also known as calcification, that is an ongoing process throughout a person's lifetime. The fibrous collagen scaffold of the bone is supplemented, and eventually outweighed, by a mineral component. This process is mediated by several internal and external influences, including the age and gender of the individual in question. There is a host of complex biological systems of promoter and inhibitory mechanisms as well as feedback from stress and strain on the bone material itself which influences mineral deposition, although the exact mechanism for the control of mineralisation level is not clear. In simple terms, when bone is subjected to mechanical stresses, microcracks may be generated, and subsequently a natural response is triggered for healing and strengthening through mineralisation.

Traditional, uncontrolled, martial arts practice for shin conditioning has many risks when inexperienced users under improper or insufficient guidance perform exercises that impose mechanical impact on areas of the shin. There is therefore a need for improvements in this field.

### SUMMARY

An object of the present invention is to provide an improved method of bone conditioning, in particular shin conditioning. Further objects include to provide a method of bone conditioning which is safe for a novice user and to provide a device for performing shin conditioning in a safe, effective, and controlled manner.

The present invention provides a method for conditioning the shin, that is characterised by an intelligent generation and execution of a treatment plan based on the state of the user so that microcracks are generated and allowed to heal in an optimal manner. The method is implemented by a device that applies localised stresses to generate microcracks on the surface of the shin. The exact application of stresses can be registered through a sensing device. The treatment plan is personalised for each user.

The present invention therefore provides a method for non-therapeutic conditioning of a user's bone. The method comprises assessing the state of the bone and generating, based upon the state of the bone, a treatment plan for conditioning the bone by forming microcracks in the bone. The method further comprises inducing localised stresses on at least part of the bone in accordance with the treatment plan. The localised stresses are induced by a conditioning device comprising a contact device and an actuator. A controller is configured to control the conditioning device in accordance with the treatment plan to mechanically induce the localised stresses on the bone.

### BRIEF DESCRIPTION OF THE DRAWINGS

By way of example only, embodiments in accordance with the present disclosure are now described with reference to, and as shown in, the accompanying drawings, in which:

FIGS. 1a and 1b are side elevational views of the lower leg illustrating the tibia (shin) and fibula;

FIG. 1c is a cross-sectional view of the lower leg showing tibia, fibula, muscles, and skin;

FIG. 2 is a schematic of a conditioning apparatus for implementing the method of the present disclosure;

FIGS. 3a, 3b and 3c are perspective views of embodiments of a conditioning device of the present disclosure;

FIGS. 4a and 4b are perspective views of embodiments of a sensing device of the conditioning apparatus of FIG. 2;

FIG. 5 is a flowchart of the method of the present disclosure;

FIG. 6 is a front perspective view of a first embodiment of a conditioning device of the present disclosure;

FIGS. 7A to 7C are top sectional views of the conditioning device of FIG. 6 showing different configurations;

FIG. 8 is a front perspective view of a further embodiment of the conditioning device of the present disclosure;

FIG. 9 is a front perspective view of a further embodiment of the conditioning device of the present disclosure;

FIG. 10 is a front perspective view of a further embodiment of the conditioning device of the present disclosure;

FIGS. 11A to 11C are a top sectional views of the conditioning device of FIG. 9 showing different configurations; and

FIG. 12 is a rear perspective view of a variation of the conditioning device of FIG. 9.

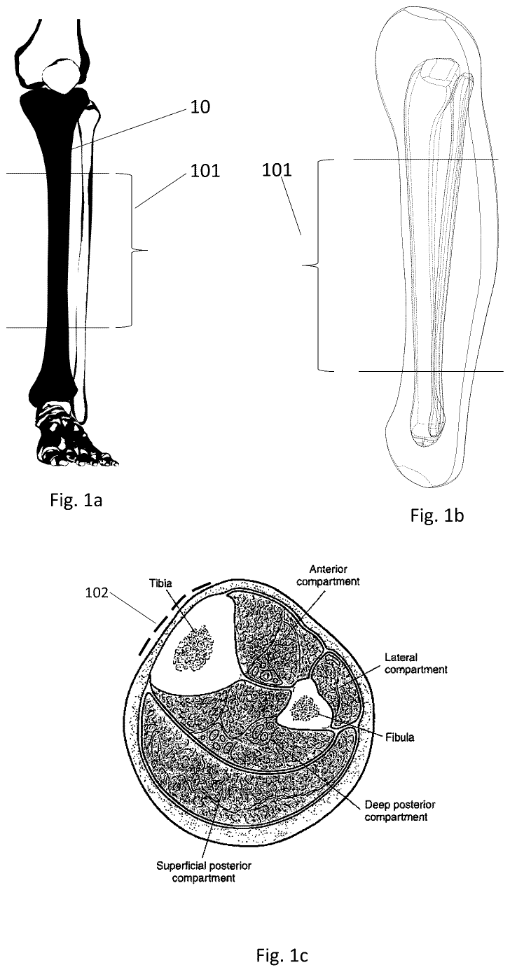

### DETAILED DESCRIPTION

The present invention relates to a method and a device for strengthening a user's bone, particularly their shin. It will be appreciated that in the present disclosure, any reference of the application of stresses or a component to the user's bone means application by contact with the skin covering the user's bone.

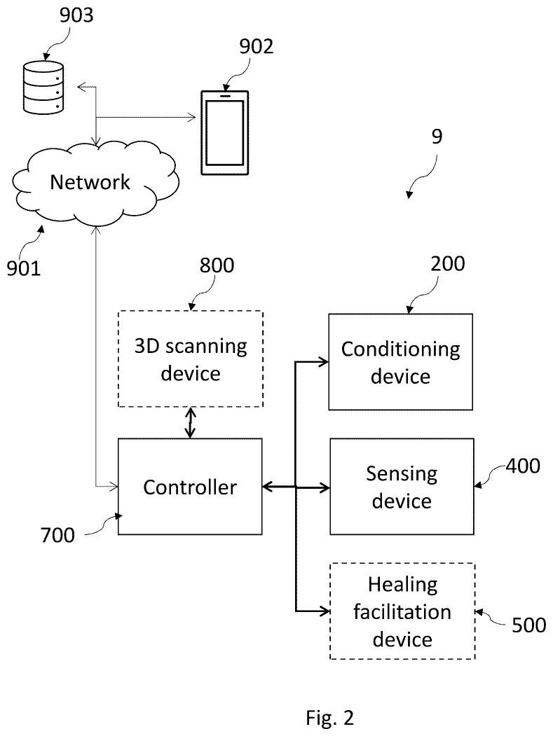

An apparatus 9 for conditioning of a user's bone 10 is disclosed herein. As shown in FIG. 2, the apparatus 9 may generally comprise a plurality of modules 200, 400, 500, 700, 800. The modules may comprise a stress applicator device 200 and a controller 700. Optional further modules may comprise a sensing device 400, a healing facilitation device 500, and a 3D scanning device 800.

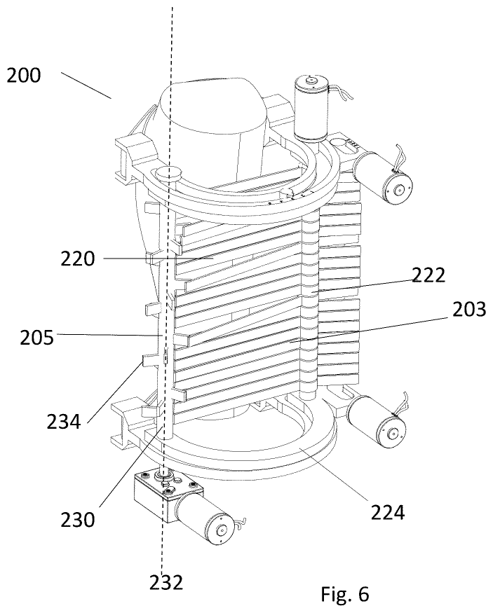

As illustrated in FIGS. 6, 9 and 10, the conditioning device 200 comprises a contact device 203 and an actuator 205. The conditioning device 200 may also be referred to as a stress applicator device. Specific embodiments of the conditioning device 200 are described in more detail below. The conditioning device 200 may also be described as a stress applicator device 200, an applicator of mechanical stresses, and/or a treatment device. The conditioning device 200 may be configured to apply localised stresses on at least part of the bone 10 in accordance with a treatment plan.

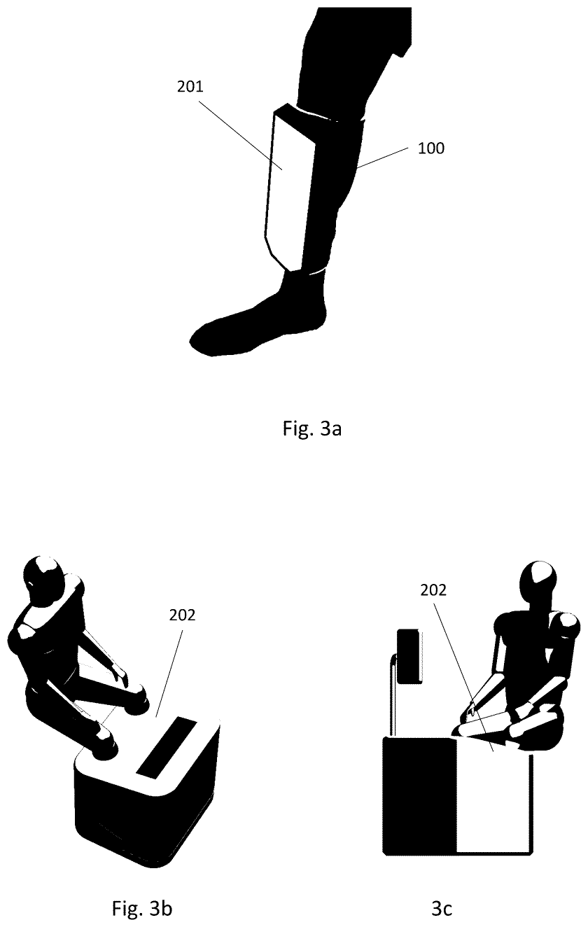

The conditioning device 200 may be a device that engages with the leg and applies mechanical stresses on the shin. As shown in FIG. 3a, the conditioning device 200 may be a wearable device 201 at least partially wrapped around, mounted to and/or secured around the lower leg 100. FIGS. 3b and 3c illustrate alternative embodiments of the conditioning device 200, comprising a stationary device 202 into which the user inserts their legs.

The sensing device 400 may sense and/or monitor the application of localised stresses, which may be mechanical, on at least part of the bone 10. The sensing device 400 may send information regarding the application of localised stresses to the controller 700 so that the treatment plan can be updated.

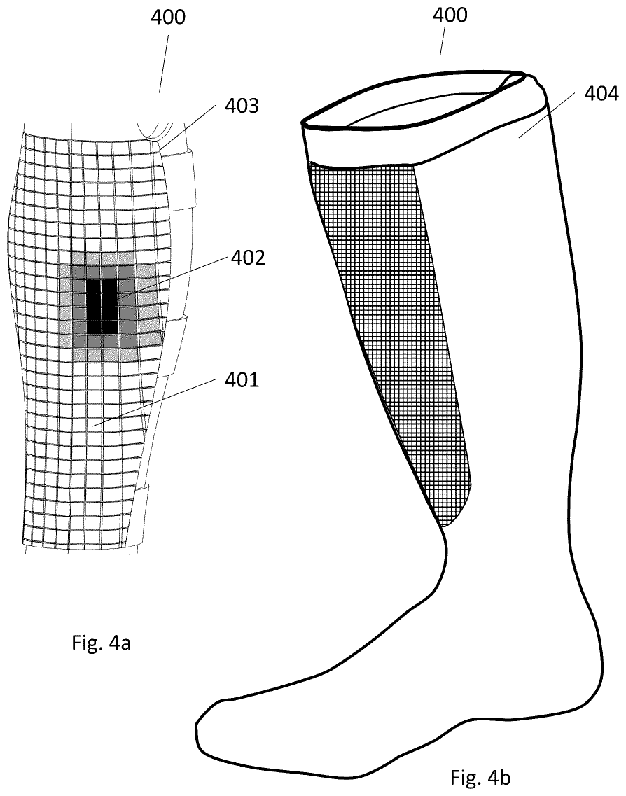

The sensing device 400 can take alternative forms as shown in FIGS. 4a and 4b. As illustrated, the sensing device 400 may comprise a matrix of sensors or sensing elements 401, configured to cover at least a portion of the user's bone and/or wearable on the shin area. The sensing device 400 may provide a precise local measurement of localized stresses applied on a shin area. Each sensing element 401 may sense and/or monitor the application of localised stresses over a cell or relatively small area of the shin area, such that the shin area is divided into a plurality of adjacent cells. Sensing by the sensing elements 401 in each of the plurality of cells enables the generation of a representation of relative magnitudes of stresses applied in the form of the heat map 402 over the shin area. The sensing device 400 may therefore generate data for forming a heat-map 402 of stresses and/or a heatmap of cumulated stress energy from the beginning of a treatment session.

The sensing device 400 may be in the form of a sleeve 403 that is wrapped around the leg as shown in FIG. 4a. The matrix of sensors may be embedded within the sleeve 403 configured to be worn on a limb of the user. In an alternative embodiment, as shown in FIG. 4b, the sensing device 400 is in the form of a wearable long sock 404and the matrix of sensors 401 may be embedded in the long sock 404.

The healing facilitation device 500 may be configured for temperature management of the users bone 10 with application or removal of heat. The healing facilitation device 500 may comprise thermal exchange elements embedded within the sensing device 400. The healing facilitation device 500 may promote the repair of microcracks so that the bone crystalline structure becomes stronger after healing.

The 3D scanning device 800 may comprise a means for obtaining an accurate 3D representation of the geometry of the user's bone 10 and/or limb. The information of the 3D geometry of the user's bone 10 may be used by the controller 700 to determine the correct or suitable location of the application of the localized stresses by the conditioning device 200. This may be important due to the different geometry of cross sections of the tibia along its length.

The apparatus 9 may comprise a position or geometric referencing device for enabling the repeated, same, positioning on the user's leg of the conditioning and/or sensing device 200, 400. The geometric referencing device may be configured to locate the conditioning and/or sensing device 200, 400 relative to at least one reference point, preferably two reference points, on the user's bone 10. The at least one reference point may be positioned on the user's bone 10 by the user prior to commencement of treatment, for example by the geometric referencing device comprising a patch or ink applied by the user. The geometric referencing device may comprise parts of the conditioning and/or sensing device 200, 400 for locating adjacent to the at least one reference point. The geometric referencing device may comprise a jig integrated or external to the conditioning and/or sensing device 200, 400. As a result, the conditioning device and/or sensing device 200, 400 may be removed and reapplied in the same position in between conditioning sessions. Precise and repeatable application of localized stresses may then be beneficially achieved in a long-term treatment plan.

The user may alternatively follow an alignment protocol to locate one at least one reference point by locating a point at the edge of the tibia towards the inner side of the leg, found four fingers below the knee. Such a simple protocol may provide a sufficiently accurate means of achieving good repeatability in the referencing of the conditioning and/or sensing device 200,400.

The apparatus 9 may comprise at least one mobile device 902 (such as a smartphone) and/or server 903 connected to the controller 700, conditioning device 200 and/or sensing device 400 via a network 901 (such as the Internet). A user may be able to control the apparatus 9 via an interface, such as a touchscreen, of the at least one mobile device 902.

The controller 700 is connected to and configured to control the conditioning device 200, particularly operational settings thereof, in accordance with the treatment plan (which may comprise a control strategy for the conditioning device 200) so as to selectively mechanically induce the localised stresses on the bone 10. The controller 700 may control the amplitude and frequency of the application of localised mechanical stresses by the conditioning device 200 according to the treatment plan. Thus mechanical stresses are optimally applied and are applied within thresholds of safety and pain. The controller 700 may also control and/or monitor the interval between treatments over a period of time.

The controller 700 may be included within the conditioning device 200 or may be separate to the conditioning device 200, such as being located on a remote server 903 or on the mobile device 902. The controller 700 may comprise a processor and memory as is well known in the art.

The controller 700 may also be configured to generate the treatment plan. The controller 700 may be connected to and configured to communicate with the remote server 903, at least one mobile device 902, sensing device 400, healing facilitation device 500, and/or the 3D scanning device 800 in order to create and update the treatment plan, as detailed further below.

The controller 700 may receive inputs regarding the state of the user and objectives of the user and use such inputs in the generation of the treatment plan. The controller 700 may also be configured to provide outputs for providing information to users. Such input(s) may be provided by the user to at least one input device, and such output(s) may be provided to at least one output device, forming part of or connected to the remote server 903, at least one mobile device 902, the controller 700 itself, the conditioning device 200 and/or the sensing device 400. In particular, the at least one input device and at least one output device may be embodied in the at least one mobile device 902, which may be configured to run an application for receiving inputs and providing outputs. The application may store personal data of the user. The personal data may comprise the state of the user, prior usage of treatment device and history of treatments, and user preferences. The application may compute the treatment plan for a treatment session, particularly where the at least mobile device 902 at least partially comprises the controller 700.

A method 300 for non-therapeutic conditioning of a user's bone 10 is disclosed herein, particularly using the aforementioned apparatus 9. The user's bone 10 may be a shin bone. However, any bone 10 can be conditioned using this method 300. The non-therapeutic conditioning may be for strengthening the user's bone (including parts thereof), the shin or parts of the shin.

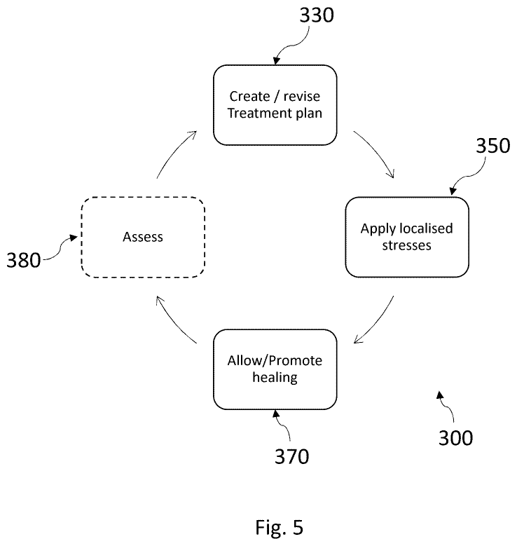

As shown in FIG. 5, the method 300 generally comprises assessing 380 the state of the bone 10; generating 330, based upon the state of the bone 10, a treatment plan for conditioning the bone 10 by forming microcracks in the bone 10; and inducing 350, by the conditioning device 200, localised stresses on at least part of the bone 10 in accordance with the treatment plan.

Assessing 380 the state of the bone 10 may be based upon a state of the user and may comprise receiving an operator defined input, which may be provided to the at least one input device, such as to an application on the mobile device 902. The operator defined input preferably includes one or more of: a specified age of the user; an experience level in martial arts; a bone conditioning history; and/or a desired intensity. The specified age of the user may be the age of the user whose bone is being conditioned and may be in the range of 1 to 99. The experience level in martial arts may be a level from 1 to 5, or 1 to 10 and may be defined for use with this method 300. The experience level in martial arts may correspond to existing categories, such as belt colour in certain martial arts disciplines. The bone conditioning history may comprise the number of times a user has previously used the conditioning device 200. The bone conditioning history may comprise data from previous bone conditioning sessions. The desired intensity may be selected from a predetermined range of levels, such as a level from 1 to 5, or 1 to 10.

The operator defined input may comprise manual inputs and automatic inputs. Manual inputs may be those inputs that a user enters manually (usually once) and comprise at least one of the following parameters: the specified age, a height of the user, a weight of the user, the experience level, previous practice of shin conditioning, and objectives of the user. Objectives of the user may be selected from predetermined list of objectives, such as “preparation for fight”, “strength building”, “strength maintenance” and so on. Automatic inputs may comprise data relating to the user that is stored on the controller 700 from previous sessions, such as being stored in non-volatile memory, including the bone conditioning history.

Assessing 380 the state of the bone 10 may comprise scanning a limb of the user comprising the bone using the 3D scanning device 800 to create the 3D model of the geometry of the limb and/or bone 10 of the user, such as of the user's leg and/or shin. The model may be stored in the memory of the controller 700, for example. The method 300 may comprise, at the controller 700, identifying areas of the 3D model at which to apply localised stresses. In the application step 350, localised stresses may then be applied on at least part of the bone corresponding to the identified areas of the 3D model.

Generating 330 the treatment plan may comprise processing data received relating to the state of the bone 10 at the controller 700, particularly from the operator defined input, using an algorithm or computer program. The operator defined input may be translated by the controller 700 into a numeric expression that the algorithm can process and take into account when creating a treatment plan. For example, the operator defined input regarding experience in martial arts may comprise a type of martial arts practiced and formal qualifications or relative scale of experience (for example scale 1 to 10). The controller 700 may utilise the algorithm to convert this information to a number that will be used.

In the creation of a treatment plan, the controller 700 may also receive operator defined input that refer directly to parameters of the treatment plan, which may effectively override automatic creation of such parameters based upon other operator defined inputs. Therefore, for example, a user may define treatment intensity and pain tolerance levels. Advanced users may also define operator defined inputs comprising intensity and frequency, where frequency corresponds to the time between successive applications of localised stresses to a local area.

The treatment plan may comprise a method of controlling the conditioning device 200 based upon the operator defined inputs. The method of controlling the conditioning device 200 may comprise adjusting operational settings of the conditioning device 200, such as at least the amplitude and/or frequency of the application of the contact device 203 by the actuator 205 on the bone 10. The amplitude and/or frequency may be selected based upon, and/or may be outputs of, the processing of the operator defined inputs in the algorithm. The algorithm may comprise a predefined map between the operator defined inputs and the operational settings and/or such links may be based upon machine learning and/or neural networks.

The treatment plan may comprise a master treatment plan that comprises several treatment plans over multiple planned sessions, and session-specific treatment plans for each specific session. The master treatment plan may be based on a treatment period (for example six months) and regular sessions (for example weekly sessions). The treatment plan may include the controller 700 controlling and/or monitoring an interval between several treatments over a period of time. The controller 700 may be configured to generate alerts to the user, such as on the at least one mobile device 902 and/or another output device, informing them that a session is due. Each treatment may be considered as one session.

The method 300 may also include, after generating the treatment plan at step 330, requiring and/or obtaining the approval of the user prior to operating of the conditioning device 200. The user confirmation may be provided after the parameters of the treatment plan are presented to the user via the at least one output device, and the user may be required to enter a confirmation via the at least one input device.. Once the user grants approval, the controller 700 may proceed to start the treatment plan. At all times the user may pause, interrupt, or overwrite the treatment plan via the at least one input device. Overwriting the treatment plan may only allow reducing the severity of the applied mechanical stresses via the at least one input device. Increasing severity may require cancellation of the treatment plan and generation of a new treatment plan. The controller 700 may create two or more alternative treatment plans and the user may choose a treatment plan of preference for that session.

Prior to application of the conditioning device 200 to the user, the method 300 includes positioning the conditioning device 200 and sensing device 400 on the user for performing the conditioning. In particular, the method 300 may include positioning the conditioning device 200 and/or sensing device 400 relative to the user, particularly their shin, limb or bone 10, using the position or geometric referencing device and/or aforementioned alignment protocol. Such a step may ensure that the conditioning device 200 and/or sensing device 400 is located in the same position as in any previous sessions and/or can be located in the same position in future sessions. Such positioning applies whether the conditioning device 200 is the wearable device 201 wearable around the lower leg as shown in FIG. 3a or is the stationary device 202 into which the legs are inserted as shown in FIGS. 3b and 3c.

The controller 700, in step 350, may then operate the conditioning device 200 to induce or apply, mechanically, localised stresses on at least part of the bone 10 in accordance with the treatment plan. The controller 700 may adjust the operational settings of the conditioning device 200 so as to adjust the localised stresses according to the treatment plan, such as by adjusting the amplitude (i.e., magnitude) and frequency of application of the localised stresses. The localised stresses, which may be referred to as localised forces, may be sufficient to create microcracks on the bone surface, particularly at the surface of the user's skin over the bone surface. The application of localised stresses may be adjusted selectively at different regions of the shin.

As discussed above, the section of the tibia bone is not the same along its length. This variation creates a non-uniform 3-dimensional shape, which the treatment plan may take into account during the application of localised stresses. Such localised stresses may be accurate, with respect to repeatability, on a small region of impact or can be randomised on a larger region of impact. The conditioning device 200 may thus apply different amplitudes and/or frequency at different parts of the bone 10 so as to induce microcracks in according with the variation in shape of the bone 10.

Subsequently, between step 350 and step 370, the conditioning device 200 may be removed from the user or the user may remove their limb from the conditioning device 200.

As shown in FIG. 5, the method 300 may further comprise at step 370 allowing/promoting healing and/or repair of the bone 10 in accordance with the treatment plan. Such healing may result in the bone crystalline structure becoming stronger and thus being conditioned. The healing step may comprise, in accordance with the treatment plan, waiting for a time interval for the promotion of the repair of the microcracks between the application of localised stresses by the conditioning device 200. After expiry of the time interval, a second set of localised stresses may be applied, in a second session, by the conditioning device on at least part of the bone in accordance with the treatment plan (i.e., as per step 350).

As illustrated in FIG. 5, the sequence may be repeated over a period of time and may be adjusted session-after-session. The method 300 may be used in a cycle in which, after the application of the localised stresses, the state of the bone 10 may be reassessed. Based upon the reassessed state of the bone 10, the treatment plan may then be updated. In particular, further operator defined inputs may be provided after a first session of application of stresses by the conditioning device 200 at step 380. The controller 700 may then adjust the treatment plan at step 330, including taking into account historic data relating to the first session of the application of stresses by the conditioning device and/or any further operator defined inputs. The controller 700 may then operate the conditioning device 200 in accordance with the updated treatment plan.

The method 300 may further comprise, during the operating of the conditioning device 200, monitoring the application of the localised stresses using the sensing device 400. The application of localised stresses may be monitored on the entire treatment surface of the bone 10 and/or shin. In preparing a session-specific treatment plan, the controller 700 may take automatic inputs from the sensing device 400. Reassessing the state of the bone, and the historic data relating to earlier sessions of the application of stresses by the conditioning device 200, may be based upon the monitored application of localised stresses.

The method 300 may therefore enable the controlled application of localised stresses so as to induce microcracks, and conditioning the user's bone, is a controlled manner. The user may control the application by adjusting the treatment plan via the user defined inputs, whether by the manual or automatic inputs. The manual inputs allow the user to manually change the treatment plan, whilst the automatic inputs allow for historic treatment sessions to be used to redefine the treatment plan as needs be. The sensing device 400 may allow for accurate monitoring of the treatment such that the treatment plan can be adjusted to take account for the monitored treatment in future.

Embodiments of the conditioning device 200 for conditioning a user's bone 10 are now described in relation to FIGS. 6 to 12. The conditioning device 200 comprises a securing means 210 for securing the conditioning device 200 to a user's body in proximity to the bone 10; a contact device 203 for mechanically inducing localised stresses on at least part of the bone 10, for forming microcracks in the bone 10; and an actuator 205 for actuating the contact device 203. The controller 700 may be configured to adjust at least the amplitude and/or frequency of the application of the contact device 203 by the actuator 205 on the bone 10 based upon the treatment plan.

The securing means 210 may comprise means for attachment to the lower leg. The securing means 210 may comprise straps or belts. The securing means 210 may be a means of installation and/or clamping of the conditioning device 200 onto the user's lower leg. The securing means 210 may allow the conditioning device 200 to be installed around a limb of the user and specifically a lower leg of the user. The securing means 210 may comprise the geometric referencing device, which may be in the form of an angular indicator for referencing zero angular position such that installation on the lower leg can be consistent each time that the apparatus is installed by the user.

In one embodiment of the conditioning device 200, shown in FIGS. 6 to 8, the contact device 203 may comprises at least one impact element 220 and the actuator 205 may be configured to operate the at least one impact element 220 to repeatedly impact the user to apply localised stresses on the part of the bone 10. The at least one impact element 220 may comprise at least one resilient member or cantilever.

The at least one resilient element 220 may comprises a plurality of resilient elements or a group of resilient elements. The at least one resilient member 220 may comprise a plurality of identical springs. Alternatively, the at least one resilient member 220 may comprise a group of springs of different characteristics, wherein the characteristic of each spring is dependent on the position of the spring in the group of springs. The at least one resilient member 220 may be a leaf spring fixed at a first end 222.

The leaf springs may each comprise a metal core, overcoated by a rubber layer. Each leaf spring may comprise a flat or planar, straight and/or thin rectangular cuboidal shape. The leaf springs may curve along their length. The leaf springs may comprise an impact shoe for contacting the user, the impact shoe having a plurality of small projections.

The at least one resilient member 220 may comprise a spring or springs wherein the springs may be compressed coil springs, stretchable in bending. The springs may be overcoated in a rubber layer.

The at least one resilient member 220 may be deformed and constrained until a point of release so that, upon release, the resilient member 220 will convert stored potential energy to kinetic energy. This will create a localised impact, force and/or stresses at a point on the shin 10 as the resilient member 220 travel comes to an abrupt stop when it encounters the shin 10.

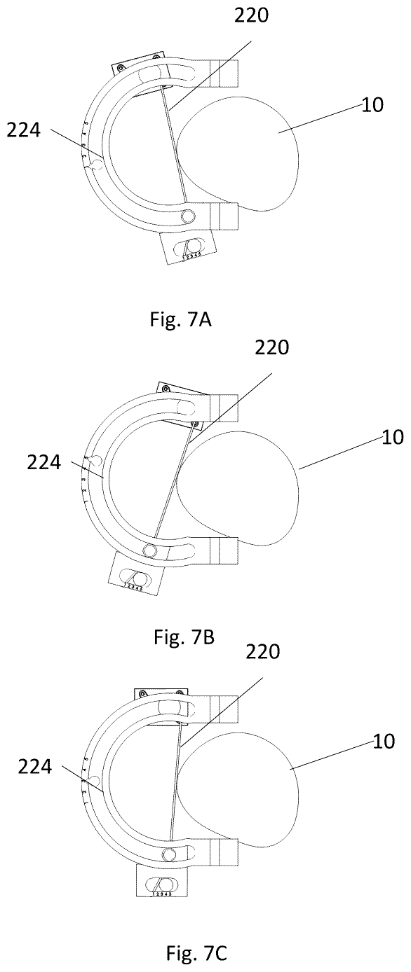

The conditioning device 200 may comprise a fastener of the at least one resilient member 220, an angle adjuster of the angle of impact of the at least one resilient member 220 upon the bone 10, and a force adjuster of the impact force. As shown in FIG. 6 and particularly FIG. 7, the angle adjuster may comprise at least one curved rail 224, which may, in use, curve at least partially around the circumference of the user's limb. The at least one resilient member 220 may be mounted to the at least one curved rail 224 such that when the resilient member 220 is moved along the at least one curved rail 224, the angle of impact, of the at least one resilient member 220 upon the bone 10, is adjusted.

As shown in FIG. 6, the actuator 205 may comprise a bar 230 extending along a longitudinal axis 232 and at least one radial projection 234. The bar 230 may be configured to rotate around the longitudinal axis 232, which may extend substantially along the height or length of the user's limb. In the embodiment of FIG. 6, the actuator 205 may be described as a rotating finger mechanism, with the bar 230 as a finger holding bar and the radial projection 234 as a finger. The at least one radial projection 234 may be configured to contact the at least one resilient element 220 to elastically deform the at least one resilient element 220 and then release the at least one resilient element 220 when the bar 230 is rotated. Thus, the stretching of the at least one resilient element 220 may be affected by the rotating finger mechanism 205. During a first point of their rotational path, the projecting fingers 234 may interact with a free tip of the at least one resilient element 220 to stretch or bias the at least one resilient element 220. During a second point of their rotational path, the projecting fingers 234 may release the free tip of the at least one resilient element 220, so that the at least one resilient element 220, after accumulating potential energy due to stretching, is able to move freely towards the shin 10, and make impact with a point on the shin 10.

In an embodiment where the at least one resilient element 220 comprises a plurality of resilient elements 220, each radial projection 234 on the rotating finger mechanism 205 may act on one corresponding resilient element of the group of resilient elements 220. Each radial projection may be offset to an adjacent radial projection by an angle (φ) wherein (φ) is between 10 and 90 degrees. Therefore, at each time window, a subset of the total resilient elements 220 in the group of resilient elements 220 is stretched and then impacts the shin 10. This creates a sequential and periodic impacting action along the length of the shin 10.

The actuator 205 may be configured to rotate the bar 230 at an angular rotational speed. The angular rotational speed may be adjustable and may controlled by the rotational speed of a motor that drives the bar 230, the motor 205 forming part of the actuator 205.

The conditioning device 200 may also comprise a resilient element-bias adjustment mechanism. The biasing of the resilient element may result in a specific static pressure when contacting the shin 10. The biasing is adjustable via the resilient element-bias adjustment mechanism. The resilient element-bias adjustment mechanism may comprise means of displacing a non-impacting end of the resilient element 220. The adjustment of the impact force on the shin 10, due to a variable degree of stretching, may also be achieved by a mechanism for adjusting the position of the bar 230. In other words, the resilient element-bias adjustment mechanism may be a mechanism which changes a position of the actuator, relative to the at least one resilient element 220, for changing a maximum deformation which the at least one resilient element 220 experiences. This may allow for adjusting a contact force applied by the at least one resilient element 220 on the user's body.

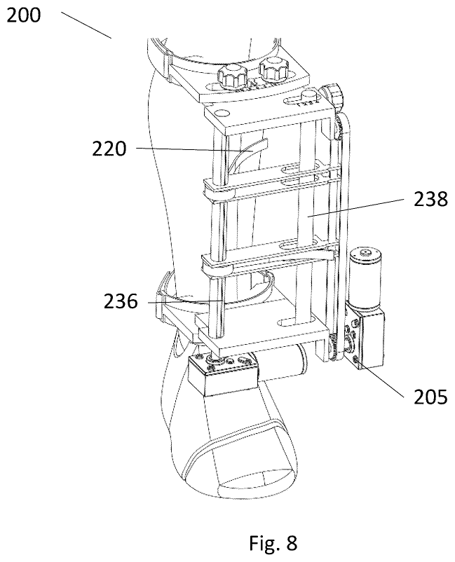

As shown in the further embodiment of the conditioning device 200 of FIG. 8, the stretching of the at least one resilient element 220 may be affected by an actuator 205 that rotates the at least one resilient element 220. The at least one resilient element 220 may be rotated by a resilient element-holding bar 236. Upon rotation, each resilient element 220 abuts a constraining bar 238 or an abutting bar. Abutment to the constraining bar 238 stretches and deforms each resilient element 220 until the deformation is sufficient to release the resilient element 220 from against the constraining bar 238. Thus, after accumulating potential energy due to stretching and deformation, each resilient element 220 is able to move freely towards the shin 10 and make impact with a point on the shin 10. This may be described as the application of localized stress using a rotating sprint structure.

The actuator 205 may be configured to adjust the rotation of the resilient element-holding bar 236 at an angular rotational speed and by controlling the rotational speed of a motor of the actuator 205 that drives the resilient element-holding bar 236.

In the embodiment of FIG. 8, each resilient element 220, in the group of resilient elements, may be offset by an angle (Θ) wherein (Θ) is between 10 and 90 degrees, so that in each time window a subset of the total number of resilient elements 220 in the group of resilient elements 220 is stretched and then impacts the shin 10. This creates a sequential and periodic impacting action along the length of the shin 10.

The adjustment of the impact force on the shin, due to a variable degree of stretching, may be achieved by a mechanism for adjusting the position of the constraining bar 238.

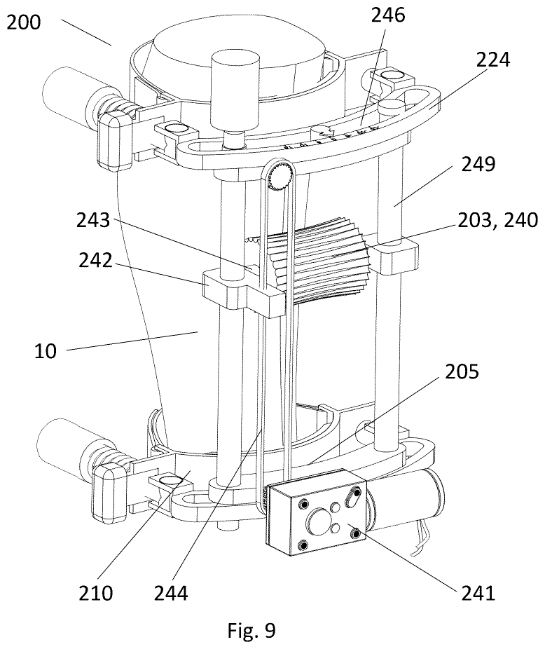

In a second embodiment of the conditioning device 200 described above, as shown in FIGS. 9 to 12, the contact device 203 may comprise at least one rolling element 240 wherein the actuator 205 is configured to move the at least one rolling element 240 such that the at least one rolling element 240 moves and/or rolls along the user's bone 10 while contacting the user to apply localised stresses on the part of the bone 10.

The conditioning device 200 may apply pressure and/or impact by utilising the rolling element 240. The rolling element 240 may be a moveable roller. The rolling element 240 may be moveable along a primary axis of the shin 10. The apparatus 9 may comprise at least one gantry or frame 426 for supporting the rolling element 240.

The at least one rolling element 240 may comprise a non-circular cross-section. The rolling element 240 may comprise projections on an outer surface or on its periphery, a rough outer surface and/or a plurality of grooves running across its width around its periphery (i.e. substantially parallel to its axis of rotation). The rolling element 240 may have an outer surface about its axis of rotation that is curved along the direction of the axis of rotation. Thus the rolling element 240 may have a smaller diameter at the centre of its width than at the edges of its width. This curved shape means the rolling element 240 more closely matches the user's shin 10, thereby causing more contact area between the rolling element 240 and users shin 10.

The at least one rolling element 240 may comprise a core and a material around core, wherein the material has surface patterns. The material around the core may be the same material as the core. Alternatively, the material around the core may be different from the material of the core. The material around the core may have a Shore hardness that is selected according to the requirements of each user. The at least one rolling element 240 may comprise a core and cylindrical members around the core. The cylindrical members may comprise wooden or bamboo members. The cylindrical members comprise extruded plastic or extruded hard rubber.

The conditioning device 200 may comprise a motion actuation mechanism to enable the rolling element 240 to change linear position relative to the shin 10 whilst the rolling element 240 also rotates about its axis of rotation. The motion actuation mechanism may comprise a means for effecting linear motion towards the gantry or towards the top of the frame 246.

The conditioning device 200 may thus comprise at least one guide pillar 249, which may form part of the motion actuation mechanism. The guide pillar 249 may form part of the gantry or frame 246. The rolling element 240 may be mounted to the at least one guide pillar 249 such that during linear movement, the rolling element 240 moves or is moved along the guide pillar 249.

Linear movement may be movement along the primary axis of the shin 10. Linear movement may be achieved in a variety of ways.

As shown in FIG. 9, the actuator 205 may comprise a motor 241, and may further comprise a belt 244 slidably mounted on the frame 246. The belt 244 may be connected to the motor 241 and the at least one rolling element 240. The motor 241 may be configured to drive the belt 244 and thereby move the at least one rolling element 240 along the user's bone 10. The motor 241 may be a stepper motor. In this embodiment, the linear movement along the primary axis of the shin 10 may be achieved by the belt 244 and the motor 241 that acts on a supporting beam 242 that is connected to a roller axle 243 of the rolling element 240. The supporting beam 242 may be slidably mounted to the guide pillar 249. This may be considered to be a roller construction comprising a wire rope structure.

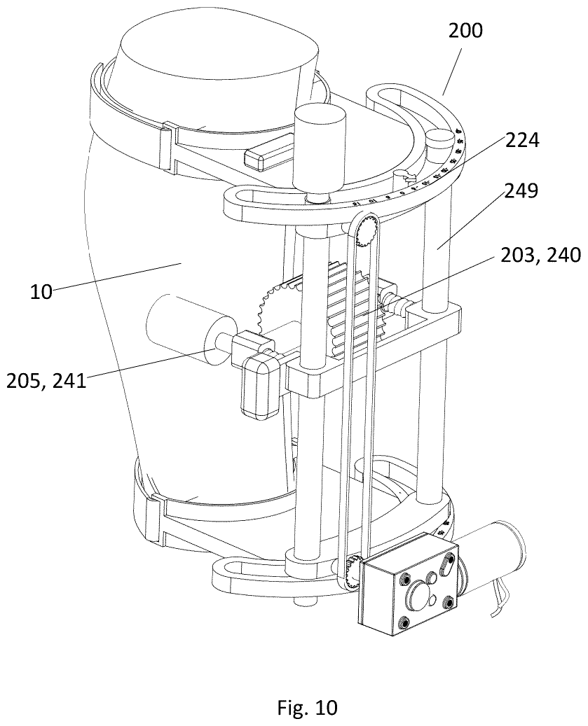

As shown in the further embodiment of the conditioning device 200 in FIG. 10, the actuator 205 may comprise a motor 241 without a belt. The motor 241 may be a hub motor that is integral to the structure of the roller 240. The hub motor may form the core of the roller 240 and the core may be fitted with a sleeve. The sleeve may comprise a urethane sleeve further comprising multiple projections. In the embodiment of FIG. 10, linear movement along the primary axis of the shin 10 may be achieved by an active rotation of the rolling element 240. Active rotation may be achieved by the motor 241.

The actuator 205 may comprise a lead strew and stepper motor (not shown). Linear movement may be achieved by the lead strew and stepper motor. The lead strew and stepper motor may act on the supporting beam 242 that is connected to the roller axle 243.

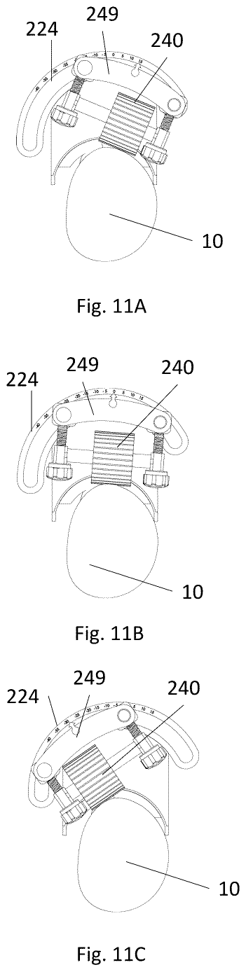

The conditioning device 200 may comprise an angular adjustment mechanism to enable the at least one rolling element 240 to change angular position relative to the shin. As shown in FIGS. 9 and 10, and most clearly in FIGS. 11A to 11C, the angular adjustment mechanism may comprise at least one curved rail 224 or an arcuate guide, which may, in use, curve at least partially around the circumference of the user's limb. The rolling element 240 may be mounted to the at least one curved rail 224 such that when the rolling element 240 is moved along the curved rail 224, the angular location of contact between the rolling element 240 and the bone 10 is adjusted. The curved rail 224 may be similar to the curved rail 224 described in connection to the first embodiment of the conditioning device.

The angular adjustment mechanism may include the guide pillars 249 sliding along and/or sliding round the curved rail 224. The guide pillars 249 may be manually rotated to slide along the curved rail 224. The guide pillars 249 may be manually secured into position by tightening screws. The guide pillars 249 may be rotated to slide along the curved rail 224 under the action of a motor to reach a pre-determined position. The pre-determined position may be determined by the controller 700.

The angular motion adjustment may take place in coordination with linear motion. Angular position may be dependent on a linear position along the primary axis (length) of the shin 10.

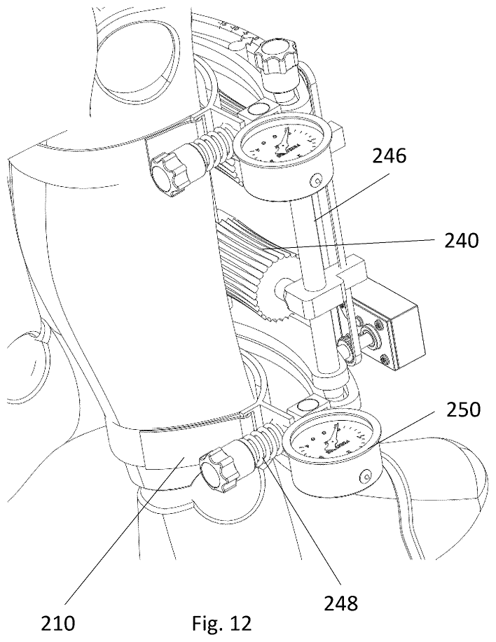

The conditioning device 200 may comprise a pressure adjustment mechanism to enable the variation of static pressure exerted by the at least one roller on the shin. The conditioning device 200 may comprise an adjustment means 248 for adjusting a contact force, a pressure and/or an impact applied by the at least one rolling element 240 on the user's body, specifically on the surface of the shin 10. As shown in FIG. 12, the adjustment means 248 may comprise at least one sprung bolt 248 mounted to the securing means 210, wherein the rolling element 240 is mounted to the at least one sprung bolt 248. The rolling element 240 may be mounted to the at least one sprung bolt 248 via the frame 246, such that the rolling element 240 is mounted to the frame 246, and the frame 246 is mounted to the at least one sprung bolt 248. The contact force, pressure and/or impact applied by the at least one rolling element 240 on the user's body may be adjusted by adjusting the sprung bolt 248.

The securing means 210 of the apparatus 9 may comprise means of securing the apparatus tightly on the lower leg. A tightness of initial placement on the lower leg may be indicated by a pressure sensing device 250 that senses initial static pressure, which may be adjusted using the pressure adjustment means 248.

The controller 700 may be configured to automatically control the position, angle, speed, and static pressure of the contact device 203. The apparatus 9 may further comprise a sensing means for sensing linear position, angular position, and static pressure of the contact device 203.

Further embodiments and examples of the invention may be found in the following numbered clauses:
A1. A method for strengthening the shin or parts of the shin, said method comprising: the preparation of a treatment plan; the application of localised stresses on at least parts of the shin, said localised stresses are mechanically induced, and sufficient to create microcracks on the bone surface, while ensuring that said localised stresses are applied to a desired surface of said at least parts of the shin, and said localised stresses are applied at a desired amplitude and frequency; and the allowance and/or promotion of the repair of said microcracks so that bone crystalline structure becomes stronger after healing, said method characterised in that a controller, comprising a processor and memory, controls the amplitude and frequency of the application of said localised mechanical stresses according to said treatment plan so that mechanical stresses are optimally applied and are within thresholds of safety and pain, and controls and/or monitors the interval of treatments over a period of time said controller receiving inputs regarding the state of the user and the objectives of the user.
A2. The method of clause A1, wherein the state of the user comprises manual inputs comprising age, experience in martial arts, previous practice of shin conditioning and automatic inputs comprising data recorded by the controller from previous sessions and stored in non-volatile memory.
A3. The method of clause A2 further comprising inputs from sensing or diagnostic devices that assess the state of the shin.
A4. The method of any preceding clause, wherein users are further able to define treatment intensity and tolerance levels.
A5. The method of any preceding clause, wherein the application of localised stresses is monitored on the entire treatment surface of the shin by a sensing device.
A6. The method of clause A5, wherein said sensing device comprises a matrix of sensors, wearable on the shin area.
A7. The method of clause A6, wherein said matrix of sensors is embedded in a long sock.
A8. The method of clause A7, wherein said matrix of sensors comprises a sleeve that is wrapped around the leg.
A9. The method of clause A5 to A8, wherein said sensing device generates a heat-map of stresses and/or a heatmap of cumulated stress energy from the beginning of the treatment session.
A10. The method of any preceding clause, wherein application of localised stresses is adjusted selectively at different regions of the shin.
A11. The method of any preceding clause, further comprising: the 3D scanning of the geometry of the user's leg, and the generation of the 3D model of the user's leg, said model stored in memory and used to generate a pattern of precise application of localised stresses.
A12. The method of any preceding clause implemented through a treatment device, said treatment device comprising: an applicator of mechanical stresses; a controller, said controller comprising a processor and memory; a sensing device for sensing localised mechanical stresses; and an input and output device for exchanging information with one or more users.
A13. The method of clause A12, said input and output device comprises means for identifying the user.
A14. The method of clause A12 or A13, said input and output device comprises a wireless connection to a smart phone and an associated application on said smart phone, wherein said application stores personal data of the user, said personal data comprising state of the user, prior usage of treatment device and history of treatments, and user preferences, and wherein said application computes treatment plan for a treatment session.
A15. The method of any of clauses A12 to A14, wherein said controller, before executing said treatment plan, requires user confirmation, wherein said user confirmation is provided after the parameters of the treatment plan are presented to the user via an output device, and the user is required to enter a confirmation via an input device.
A16. The method of any of clauses A12 to A15, said treatment device further comprises means of position referencing with respect to the geometry of the user's leg, said position referencing is applied to sensing device and to the device for application of mechanical stresses.
A17. The method of any of clauses A12 to A16, said geometric referencing comprises locating of two reference points on the shin, said reference points are positioned on the user's leg by the user prior to commencement of treatment, wherein the user follows a pre-defined protocol and/or jig to position these reference points.
B1. A method for strengthening the shin or parts of the shin in a way that simulates martial arts practices, said method comprises: mechanically inducing localised stresses on at least parts of the shin sufficient to create microcracks on the bone surface, and ensuring that said localised stresses are applied to a desired surface of said at least parts of the shin, characterised in that said stresses are applied by an apparatus that is installed around the lower leg, said apparatus applying impact forces by utilising a group of spring elements, each spring in said of group of spring elements is deformed and constrained until a point of release so that upon release, each spring of said group of spring elements will convert the stored potential energy to kinetic energy and will create an impact to a point on the shin as it's travel comes to an abrupt stop when it encounters the shin.
B2. The method of clause B1 wherein the apparatus comprises means for attachment to the lower leg, means for holding group of spring elements, means of adjusting the angle of impact of the springs, and means of adjusting the impact force.
B3. The method of clause B2, wherein the group of spring elements comprises a plurality of identical springs.
B4. The method of clause B2, wherein the group of spring elements comprises springs of different characteristics, wherein the characteristic of each spring is dependent on the position of the spring in the group of springs.
B5. The method of clause B1 to B4, wherein the springs comprise leaf springs.
B 6. The method of clause B5, wherein a width of each of said leaf springs is between 2 mm and 15 mm.
B7. The method of clause B5 or B6, wherein the leaf springs comprise a metal core, overcoated by a rubber layer.
B8. The method of any of clauses B5 to B7, wherein said leaf springs have a flat straight shape.
B9. The method of any of clauses B5 to B7, said leaf springs have a curved shape.
B10. The method of any of clauses B5 to B9, said leaf springs further comprise an impact shoe, said impact show having a plurality of small projections.
B11. The method of any of clauses B1 to B10, wherein the springs comprise a compressed coil spring stretchable in bending.
B12. The method of clause B11, wherein the springs are overcoated in a rubber layer.
B13. The method of any of clauses B1 to B12, wherein the stretching of said springs is effected by a rotating finger mechanism, said rotating finger mechanism comprising a finger-holding bar and a plurality of projecting fingers wherein the fingers rotate, and during first point of their rotational path interact with the free tip of a spring to stretch said spring, and during a second point of their rotational path release said free tip of a spring, so that said spring, after accumulating potential energy due to stretching, is able to move freely towards the shin, and make impact with a point on the shin.
B14. The method of clause B15, wherein each of said finders on said rotating finger mechanism is acting on one corresponding spring in the group of springs, and wherein each finger is offset by the previous one by an angle (φ) wherein (φ) is between 10 and 90 degrees so that at each time window a subset of the total number of springs in the group of springs is stretched and then impacting the shin, thus creating a sequential and periodic impacting action along the length of the shin.
B15. The method of clause B13 or B14, wherein the angular rotational speed of said rotating finger mechanism is adjustable and is controlled by the rotational speed of a motor that drives said finger-holding bar.
B16. The method of any of clauses B13 to B15, wherein the biasing of said springs that results in a static pressure when contacting the shin, is adjustable via a spring-bias adjustment mechanism, wherein said spring-bias adjustment mechanism comprises means of displacing the non-impacting end of said springs.
B17. The method of any of clauses B13 to B15, wherein the adjustment of the impact force on the shin, due to a variable degree of stretching, is achieved by a mechanism for adjusting the position of the abutting bar.
B18. The method of any of clauses B1 to B17, wherein stretching of said springs is effected by mechanism that rotates said springs so that upon rotation, each of said springs abuts a constraining bar, abutment to the constraining bar stretches and deforms each of said springs until the deformation is sufficient to release each of said springs against the abutting bar, so that said spring, after accumulating potential energy due to stretching and deformation, each of said springs is able to move freely towards the shin, and make impact with a point on the shin.
B19. The method of clause B18, wherein each of said springs in the group of springs is offset by an angle (Θ) wherein (Θ) is between 10 and 90 degrees, so that at each time window a subset of the total number of springs in the group of springs is stretched and then impacting the shin, thus creating a sequential and periodic impacting action along the length of the shin.
B20. The method of clause B18 or B19, wherein the angular rotational speed of said spring-holding bar is adjustable and is controlled by the rotational speed of a motor that drives said spring-holding bar.
B21. The method of any of clauses B18 to B20, wherein the adjustment of the impact force on the shin, due to a variable degree of stretching, is achieved by a mechanism for adjusting the position of the abutting bar.
C1. A method for strengthening the shin or parts of the shin in a way that simulates martial arts practices, said method comprises: mechanically inducing localised stresses on at least parts of the shin; sufficient to create microcracks on the bone surface; and ensuring that said localised stresses are applied to a desired surface of said at least parts of the shin, characterised in that said stresses are applied by an apparatus that is installed around the lower leg, said apparatus applying a combination of pressure and impact by utilising at least one movable roller, said at least one roller is movable along the primary axis of the shin, said roller has protrusions on its periphery, and said apparatus further comprises means to adjust pressure and/or impact at the surface of the shin.
C2. The method of clause C1, wherein the rollers have a curved surface.
C3. The method of clause C1 or C2, wherein the rollers comprise a core and a material around core, said material has surface patterns.
C4. The method of any of clauses C1 to C3, wherein the material around the core is the same material as the core.
C5. The method of any of clauses C1 to C3, wherein the material around the core is different from the material of the core and has shore hardness that is selected according to the requirements of each user.
C6. The method of any of clauses C1 to C5, wherein the roller structure comprises a core and cylindrical members around the core.
C7. The method of clause C6, wherein the cylindrical members comprise wooded or bamboo members.
C8. The method of clause C6, wherein the cylindrical members comprise extruded plastic or extruded hard rubber.
C9. The method of any of clauses C1 to C8, wherein said rollers construction comprises a wire rope structure.
C10. The method of any of clauses C1 to C9, wherein linear movement along the primary axis of the shin is achieved by a lead strew and stepper motor that acts on a supporting beam that is connected to the roller axle.
C11. The method of any of clauses C1 to C9, wherein linear movement along the primary axis of the shin is achieved by a belt and stepper motor that acts on a supporting beam that is connected to the roller axle.
C12. The method of any of clauses C1 to C9, wherein linear movement along the primary axis of the shin is achieved by an active rotation of the roller.
C13. The method of clause C12, wherein said active rotation is achieved by a hub motor that is integral to the structure of the roller, said hub roller forming the core of said roller and wherein said core is fitted with a sleeve.
C14. The method of clause C13, wherein said sleeve comprises a urethane sleeve further comprising multiple projections.
C15. The method of any of clauses C1 to C14, wherein the apparatus comprises: at least one roller; at least one gantry for supporting said at least one roller; a motion actuation mechanism to enable the at least one roller to change linear position relative to the shin; an angular adjustment mechanism to enable the at least one roller to change angular position relative to the shin; a pressure adjustment mechanism to enable the variation of static pressure exerted by the at least one roller on the shin; and a means of installation and clamping of the apparatus onto a user's lower leg.
C16. The method of clause C15 further comprising a controller to automatically control position, angle, speed, and static pressure.
C17. The method of clause C15 or C16, further comprising sensing means for sensing linear position, angular position, and static pressure.
C18. The method of any of clauses C15 to C17, the linear motion actuation comprising a motor and a belt mechanism for effecting linear motion to said gantry.
C19. The method of any of clauses C15 to C17, the linear motion actuation comprises a motor and lead screw for effecting linear motion to said gantry.
C20. The method of any of clauses C15 to C19, wherein the angular adjustment mechanism comprises an arcuate guide along which guide pillars can slide, said guide pillars can be manually rotated to slide along said arcuate guide and secured into position by tightening screws.
C21. The method of any of clauses C15 to C20, wherein the angular adjustment mechanism comprises an arcuate guide along which guide pillars can slide, said guide pillars are rotated to slide along said arcuate guide, under the action of a motor to reach a position that is determined by said controller.
C22. The method of any of clauses C15 to C21, wherein the angular motion adjustment takes place in coordination with linear motion, and angular position is dependent on linear position along primarily axis (length) of the shin.
C23. The method of any of clauses C15 to C22, further comprising means of installation and clamping on the lower leg, said means of installation further comprise an angular indicator for reference zero angular position such that installation on the lower leg can be consistent each time that the apparatus is installed by the user and said means of installation further comprise means of securing the apparatus tightly on the lower leg.
C24. The method of any of clauses C15 to C23, wherein tightness of initial placement on the lower leg is indicated by a pressure sensing device that senses initial static pressure, said static pressure that be adjusted by adjustment means

1. A method for non-therapeutic conditioning of a user's bone, the method comprising:
assessing a state of the bone;
generating, based upon the state of the bone, a treatment plan for conditioning the bone by forming microcracks in the bone; and
inducing, by a conditioning device comprising a contact device and an actuator, localised stresses on at least part of the bone in accordance with the treatment plan, wherein a controller is configured to control the conditioning device in accordance with the treatment plan to mechanically induce the localised stresses on the bone.

2. The method of claim 1, wherein the treatment plan comprises a method of controlling the conditioning device, the method of controlling the conditioning device comprising adjusting at least an amplitude and/or frequency of the application of the contact device by the actuator on the bone in accordance with the treatment plan.

3. The method of claim 1, wherein assessing the state of the bone comprises using an operator defined input, wherein the operator defined input preferably includes one or more of:
a specified age of the user;
an experience level in martial arts;
a bone conditioning history; and/or
a desired intensity.

4. The method of claim 1, further comprising, in accordance with the treatment plan:
waiting for a time interval for promotion of a repair of said microcracks; and
applying by the conditioning device, after expiry of the time interval, a second set of localised stresses on at least part of the bone in accordance with the treatment plan.

5. The method of claim 1 further comprising, after an application of the localised stresses, reassessing the state of the bone and updating, based upon the reassessed state of the bone, the treatment plan.

6. The method of claim 1 further comprising monitoring an application of the localised stresses using a sensing device.

7. The method of claim 6, further comprising:
reassessing the state of the bone based upon the monitored application of localised stresses, and
updating the treatment plan based upon the reassessed state of the bone.

8. The method of claim 1, further comprising:
scanning a limb of the user comprising the bone to create a 3D model of the limb of the user;
identifying areas on the 3D model on which to apply localised stresses; and
applying localised stresses on at least part of the bone corresponding to the identified areas of the 3D model.

9. The method of claim 1, wherein the user's bone is a shin bone.

10. A conditioning apparatus for conditioning of a user's bone, the conditioning apparatus comprising a conditioning device, the conditioning device comprising:
a securing means for securing the conditioning device to a user's body in proximity to the bone;
a contact device for mechanically inducing localised stresses on at least part of the bone for forming microcracks in the bone; and
an actuator for actuating the contact device.

11. The conditioning apparatus of claim 10, further comprising a controller configured to control the conditioning device in accordance with a treatment plan, the controller being configured to adjust at least the amplitude and/or frequency of the application of the contact device by the actuator on the bone based upon the treatment plan.

12. The conditioning apparatus of claim 10, wherein the contact device comprises at least one impact element and the actuator is configured to operate the at least one impact element to repeatedly impact the user to apply localised stresses on the part of the bone.

13. The conditioning apparatus of claim 12, wherein the at least one impact element comprises at least one resilient member, optionally wherein the at least one resilient member is a leaf spring fixed at a first end.

14. The conditioning apparatus of claim 13, wherein the actuator comprises a bar extending along a longitudinal axis and at least one radial projection, wherein
the bar is configured to rotate around the longitudinal axis, and
the least one radial projection is configured to contact the at least one resilient element to elastically deform the at least one resilient element and then release the at least one resilient element when the bar is rotated.

15. The conditioning apparatus of claim 13, wherein an actuator position can be changed relative to the at least one resilient element for changing a maximum deformation which the at least one resilient element experiences for adjusting a contact force applied by the at least one resilient element on the user's body.

16. The conditioning apparatus of claim 10, wherein the contact device comprises at least one rolling element and the actuator is configured to move the at least one rolling element such that the at least one rolling element moves along the user's bone while contacting the user to apply localised stresses on the part of the bone.

17. The conditioning apparatus of claim 16, wherein the at least one rolling element comprises a non-circular cross-section.

18. The conditioning apparatus of claim 16 further comprising an adjustment means for adjusting a contact force applied by the at least one rolling element on the user's body.

19. The conditioning apparatus of claim 16 wherein the actuator comprises a motor.

20. The conditioning apparatus of claim 19, wherein the actuator further comprises a belt slidably mounted on a frame and the belt is connected to the motor and the at least one rolling element, wherein the motor is configured to drive the belt and thereby move the at least one rolling element along the user's bone.

21. The conditioning apparatus of claim 10, further comprising a sensing device for monitoring the application of the localised stresses on the user's bone.

22. The conditioning apparatus of claim 21, wherein the sensing device comprises a matrix of sensors configured to cover at least a portion of the user's bone, preferably wherein the matrix of sensors is embedded within a sleeve configured to be worn on a limb of the user.

23. The conditioning apparatus of claim 10, wherein the user's bone is a shin bone.

24. The conditioning apparatus of claim 1, wherein the apparatus is applied in a method of non-therapeutic conditioning of a user's bone.
